(19) United States
(12) Patent Application Publication
(10) Pub. No.: US 2021/0369778 A1
(43) Pub. Date: Dec. 2, 2021

(54) SYSTEM AND METHOD FOR COLD ATMOSPHERIC PLASMA BASED T CELL THERAPY

(71) Applicant: Jerome Canady Research Institute for Advanced Biological and Technological Sciences, Takoma Park, MD (US)

(72) Inventors: Jerome Canady, Lakeland, FL (US); Saravana Murthy, Owings Mills, MD (US); Taisen Zhuang, Rockville, MD (US); Xiaoqian Cheng, Falls Church, VA (US)

(21) Appl. No.: 17/336,233

(22) Filed: Jun. 1, 2021

Related U.S. Application Data
(60) Provisional application No. 63/031,964, filed on May 29, 2020.

(51) Int. Cl. A61K 35/17; C12N 15/11

(52) U.S. Cl. CPC A61K 35/17; C12N 2310/20; C12N 15/111

(57) ABSTRACT

A method for using Cold Atmospheric Plasma based T cell therapy for treating cancer including treating tumor cells with cold atmospheric plasma to permeabilize the tumor cells, condensing a mammalian CRISPR plasmid vector into compact nanostructures using one of poly-L-lysine (PLL) and Star-shaped poly(ethylene glycol)-block-polyethylenimine, and transferring into the tumor cells catalytically inactive Cas9 (dCas9) containing transcriptional activators (VP64-p65-Rta) paired with single guide RNAs (sgRNAs) and plasmid DNA (CRISPRa system) to elicit immune responses by enhancing the presentation of tumor-associated antigens.

### CROSS-REFERENCE TO RELATED APPLICATIONS

The present application claims the benefit of the filing date of U.S. Provisional Patent Application Ser. No. 63/031,964 filed by the present inventors on May 29, 2020.

The aforementioned provisional patent application is hereby incorporated by reference in its entirety.

### STATEMENT REGARDING FEDERALLY SPONSORED RESEARCH OR DEVELOPMENT

None.

### BACKGROUND OF THE INVENTION

### Field of the Invention

The present invention relates to systems and methods for treating cancer with cold atmospheric plasma. More specifically, the present invention is a system and method for treating cancer with cold atmospheric plasma T cell therapy.

### Brief Description of the Related Art

Recent progress in atmospheric plasmas led to creation of cold plasmas with ion temperatures close to room temperature. Cold non-thermal atmospheric plasmas can have tremendous applications in biomedical technology. K. H. Becker, K. H. Shoenbach and J. G. Eden “Microplasma and applications” J. Phys. D.: Appl. Phys. 39, R55-R70 (2006). In particular, plasma treatment can potentially offer a minimum-invasive surgery that allows specific cell removal without influencing the whole tissue. Conventional laser surgery is based on thermal interaction and leads to accidental cell death i.e. necrosis and may cause permanent tissue damage. In contrast, non-thermal plasma interaction with tissue may allow specific cell removal without necrosis. In particular, these interactions include cell detachment without affecting cell viability, controllable cell death etc. It can be used also for cosmetic methods of regenerating the reticular architecture of the dermis. The aim of plasma interaction with tissue is not to denaturate the tissue but rather to operate under the threshold of thermal damage and to induce chemically specific response or modification. In particular presence of the plasma can promote chemical reaction that would have desired effect. Chemical reaction can be promoted by tuning the pressure, gas composition and energy. Thus, the important issues are to find conditions that produce effect on tissue without thermal treatment. Overall plasma treatment offers the advantage that is can never be thought of in most advanced laser surgery. E. Stoffels, I. E Kieft, R. E. J Sladek, L. J. M van den Bedem, E. P van der Laan, M. Steinbuch “Plasma needle for in vivo medical treatment: recent developments and perspectives” Plasma Sources Sci. Technol. 15, S169-S180 (2006).

Several different systems and methods for performing Cold Atmospheric Plasma (CAP) treatment have been disclosed. For example, U.S. Pat. No. 10,213,614 discloses a two-electrode system for CAP treatment. U.S. Pat. Nos. 9,999,462 and 10,023,858 each disclose a converter unit for using a traditional electrosurgical system with a single electrode CAP accessory to perform CAP treatment. WO 2018191265A1 disclosed an integrated electrosurgical generator and gas control module for performing CAP.

As a near-room temperature ionized gas, cold atmospheric plasma (CAP) has demonstrated its promising capability in cancer treatment by causing the selective death of cancer cells in vitro. See, Yan D, Sherman J H and Keidar M, “Cold atmospheric plasma, a novel promising anti-cancer treatment modality,” Oncotarget. 8 15977-15995 (2017); Keidar M, “Plasma for cancer treatment,” Plasma Sources Sci. Technol. 24 33001 (2015); Hirst A M, Frame F M, Arya M, Maitland N J and O'Connell D, “Low temperature plasmas as emerging cancer therapeutics: the state of play and thoughts for the future,” Tumor Biol. 37 7021-7031 (2016). The CAP treatment on several subcutaneous xenograft tumors and melanoma in mice has also demonstrated its potential clinical application. See, Keidar M, Walk R, Shashurin A, Srinivasan P, Sandler A, Dasgupta S, Ravi R, Guerrero-Preston R and Trink B, “Cold plasma selectivity and the possibility of a paradigm shift in cancer therapy,” Br. J. Cancer. 105 1295-301 (2011); Vandamme M, Robert E, Dozias S, Sobilo J, Lerondel S, Le Pape A and Pouvesle J-M, “Response of human glioma U87 xenografted on mice to non thermal plasma treatment,” Plasma Med. 1 27-43 (2011); Brulle L, Vandamme M, Ries D, Martel E, Robert E, Lerondel S, Trichet V, Richard S, Pouvesle J M and Le Pape A, “Effects of a Non thermal plasma treatment alone or in combination with gemcitabine in a MIA PaCa2-luc orthotopic pancreatic carcinoma model,” PLoS One. 7 e52653 (2012); and Chernets N, Kurpad D S, Alexeev V, Rodrigues D B and Freeman T A, “Reaction chemistry generated by nanosecond pulsed dielectric barrier discharge treatment is responsible for the tumor eradication in the B16 melanoma mouse model,” Plasma Process. Polym. 12 1400-1409 (2015).

Cancer cells have shown specific vulnerabilities to CAP. See, Yan D, Talbot A, Nourmohammadi N, Cheng X, Canady J, Sherman J and Keidar M, “Principles of using cold atmospheric plasma stimulated media for cancer treatment,” Sci. Rep. 5 18339 (2015)

Understanding the vulnerability of cancer cells to CAP will provide key guidelines for its application in cancer treatment. Only two general trends about the cancer cells' vulnerability to CAP treatment have been observed in vitro based on just a few cell lines. First, one study just compared the cytotoxicity of CAP treatment on the cancer cell lines expressing p53 with the same treatment on the cancer cell lines without expressing p53. The cancer cells expressing the p53 gene were shown to be more resistant to CAP treatment than p53 minus cancer cells. Ma Y, Ha C S, Hwang S W, Lee H J, Kim G C, Lee K W and Song K, “Non-thermal atmospheric pressure plasma preferentially induces apoptosis in p53-mutated cancer cells by activating ROS stress-response pathways,” PLoS One. 9 e91947 (2014). p53, a key tumor suppressor gene, not only restricts abnormal cells via the induction of growth arrest or apoptosis, but also protects the genome from the oxidative damage of ROS such as H₂O₂ through regulating the intracellular redox state. Sablina A A, Budanov A V, Ilyinskaya G V, Larissa S, Kravchenko J E and Chumakov P M, “The antioxidant function of the p53 tumor suppressor,” Nat. Med. 11 1306 (2005). p53 is an upstream regulator of the expression of many anti-oxidant enzymes such as glutathione peroxidase (GPX), glutaredoxin 3 (Grx3), and manganese superoxide dismutase (MnSOD). Maillet A and Pervaiz S, “Redox regulation of p53, redox effectors regulated by p53: a subtle balance,” Antioxid. Redox Signal. 16 1285-1294 (2012). In addition, the cancer cells with a lower proliferation rate are more resistant to CAP than cancer cells with a higher proliferation rate. Naciri M, Dowling D and Al-Rubeai M, “Differential sensitivity of mammalian cell lines to non-thermal atmospheric plasma,” Plasma Process. Polym. 11 391-400 (2014). This trend may be due to the general observation that the loss of p53 is a key step during tumorigenesis. Tumors at a high tumorigenic stage are more likely to have lost p53. See, Fearon E F and Vogelstein B, “A genetic model for colorectal tumorigenesis,” Cell. 61 759-767 (1990).

About 20% of invasive breast carcinomas show overexpression of human epidermal growth factor receptor type 2 (HER2), and patients with HER2-positive tumors have a decreased overall survival rate. Slamon, D., et al., Human breast cancer: correlation of relapse and survival with amplification of the HER-2/neu oncogene. Science, 1987. 235(4785): p. 177-182.

Wang et al. demonstrated that breast cancer cell line MDA-MB-231 is more sensitive to cold plasma treatment than mesenchymal stem cells (MSC) under the plasma dose conditions tested. Wang, M., et al., Cold atmospheric plasma for selectively ablating metastatic breast cancer cells. PLoS One, 2013. 8(9): p. e73741. The migration and invasion of MDA-MB-231 cells are inhibited by cold plasma treatment. Kim et al. studied the breast cell line MCF-7 treated by a pulsed atmospheric cold plasma jet, showing that the apoptotic effect is dependent on the components of plasma plume. Kim, S. J., et al., Induction of apoptosis in human breast cancer cells by a pulsed atmospheric pressure plasma jet. Applied Physics Letters, 2010. 97(2): p. 023702.

Clustered regularly interspaced short palindromic repeats (CRISPR) are a distinctive feature of the genomes of most Bacteria and Archaea and are thought to be involved in resistance to bacteriophages. See, Barrangou R, Fremaux C, Deveau H, et al. CRISPR provides acquired resistance against viruses in prokaryotes. Science. 2007; 315(5819):1709-1712; Lin J, Feng M, Zhang H, She Q. Characterization of a novel type III CRISPR-Cas effector provides new insights into the allosteric activation and suppression of the Cas10 DNase. Cell Discov. 2020; 6:29. Published 2020 May 12; and Fajrial A K, He Q Q, Wirusanti N I, Slansky J E, Ding X. A review of emerging physical transfection methods for CRISPR/Cas9-mediated gene editing. Theranostics. 2020; 10(12):5532-5549. Published 2020 Apr. 15.

### SUMMARY OF THE INVENTION

In a preferred embodiment, the present invention is a novel treatment approach for cancer using Cold Atmospheric Plasma. More specifically, the present invention is a method for using Cold Atmospheric Plasma T cell therapy to treat cancer. The method for using Cold Atmospheric Plasma based T cell therapy for treating cancer includes treating tumor cells with cold atmospheric plasma to permeabilize the tumor cells, condensing a mammalian CRISPR plasmid vector into compact nanostructures using one of poly-L-lysine (PLL) and Star-shaped poly(ethylene glycol)-block-polyethylenimine, and transferring into the tumor cells catalytically inactive Cas9 (dCas9) containing transcriptional activators (VP64-p65-Rta) paired with single guide RNAs (sgRNAs) and plasmid DNA (CRISPRa system) to elicit immune responses by enhancing the presentation of tumor-associated antigens.

Still other aspects, features, and advantages of the present invention are readily apparent from the following detailed description, simply by illustrating a preferable embodiments and implementations. The present invention is also capable of other and different embodiments and its several details can be modified in various obvious respects, all without departing from the spirit and scope of the present invention. Accordingly, the drawings and descriptions are to be regarded as illustrative in nature, and not as restrictive. Additional objects and advantages of the invention will be set forth in part in the description which follows and in part will be obvious from the description or may be learned by practice of the invention.

### BRIEF DESCRIPTION OF THE DRAWINGS

For a more complete understanding of the present invention and the advantages thereof, reference is now made to the following description and the accompanying drawings, in which:

FIG. 1 is diagram illustrating a method for using cold atmospheric plasma based T cell therapy to treat cancer in accordance with a preferred embodiment of the present invention.

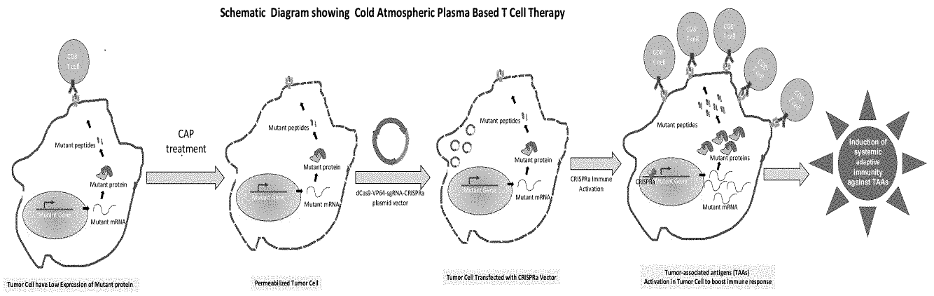

### DETAILED DESCRIPTION OF THE PREFERRED EMBODIMENTS

Recently, immunotherapy such as checkpoint blockade, adoptive cell transfer, human recombinant cytokines and cancer vaccines have shown very encouraging signs for cancer treatment, however only a subset of patients show complete response to these treatments. The principle of cancer immunotherapy is based on the identification of tumor-associated antigens (TAAs) which are dysregulated mutated gene products that are presented as antigens and neutralization of these cells by engineered T cells. However, the sparse expression of these antigens and loss of neoantigen during malignancy are insufficient to prompt a full-blown immune response to neutralize the tumor.

CRISPR activation (CRISPRa)-mediated multiplexed activation of endogenous genes as an immunotherapy (MAEGI), which acts by directly augmenting the expression and presentation of endogenous genes that encode potentially immunogenic antigens, that elicits antitumor immune responses by recruiting effector T cells and remodeling the tumor microenvironment. In this CRISPRa system viral vectors are used as a vehicle for DNA transfer into the cells. However, several studies have highlighted major drawbacks to using adenovirus as vaccine and gene therapy vectors. These include pre-existing immunity in humans, inflammatory responses, sequestering of the vector to liver and spleen, and immunodominance of the vector genes over transgenes. In previous studies of plasma mediated gene delivery has shown utility in vitro and in vivo for both drugs and genes (PMID: 17947153, PMID: 25455213).

Under controlled conditions Cold Atmospheric Plasma (CAP) temporarily permeabilizes cells to extracellular materials (M Leduc et al., 2009 New Journal of Physics) however, the size of the plasmid DNA was a limitation with just 5.5 Kb plasmid. The mammalian CRISPR plasmid vector required for MAEGI system is >10 Kb in size. Here we propose to permeabilize the tumor cells in vitro or in vivo using CAP. See, U.S. Pat. No. 9,999,462. We will condense the mammalian CRISPR plasmid vector into compact nanostructures using poly-L-lysine (PLL) (PMID: 18068848) or Star-shaped poly(ethylene glycol)-block-polyethylenimine [star-(PEG-b-PEI)](PMID: 12217037). To elicit immune responses by enhancing the presentation of TAAs, catalytically inactive Cas9 (dCas9) containing transcriptional activators (VP64-p65-Rta) paired with single guide RNAs (sgRNAs) (PMID: 23452860; PMID: 25730490) plasmid DNA (CRISPRa system) would be transferred into the cells after treating the cells with CAP by CCPCS and permeabilize the tumor cells. The specific TAAs will be selected depending on the tumor cell type. The expression and presentation of endogenous genes that encode potentially immunogenic antigens (TAA) will be activated by CRISPRa system. This in turn will induce stronger presentation and the immune response mediated by the T cells which recognize and kill tumor cells. This would further prime the activation of more T cells and suppress/completely eliminate the tumor cells.

The foregoing description of the preferred embodiment of the invention has been presented for purposes of illustration and description. It is not intended to be exhaustive or to limit the invention to the precise form disclosed, and modifications and variations are possible in light of the above teachings or may be acquired from practice of the invention. The embodiment was chosen and described in order to explain the principles of the invention and its practical application to enable one skilled in the art to utilize the invention in various embodiments as are suited to the particular use contemplated. It is intended that the scope of the invention be defined by the claims appended hereto, and their equivalents. The entirety of each of the aforementioned documents is incorporated by reference herein.

What is claimed is:

1. A method for using Cold Atmospheric Plasma based T cell therapy for treating cancer, comprising:
treating tumor cells with cold atmospheric plasma to permeabilize the tumor cells;
condensing a mammalian CRISPR plasmid vector into compact nanostructures using one of poly-L-lysine (PLL) and Star-shaped poly(ethylene glycol)-block-polyethylenimine; and
transferring into the tumor cells catalytically inactive Cas9 (dCas9) containing transcriptional activators (VP64-p65-Rta) paired with single guide RNAs (sgRNAs) and plasmid DNA (CRISPRa system) to elicit immune responses by enhancing the presentation of tumor-associated antigens.

2. A method according to claim 1, wherein the tumor cells are treating with cold atmospheric plasma in vitro.

3. A method according to claim 1, wherein the tumor cells are treating with cold atmospheric plasma in vivo.
